(19) United States
(12) Patent Application Publication
(10) Pub. No.: US 2009/0165183 A1
(43) Pub. Date: Jul. 2, 2009

(54) HEAT EXCHANGE GARMENT

(76) Inventor: Andrew Robert England Kerr, Birmingham (GB)

Correspondence Address: HAHN & VOIGHT PLLC, 1012 14TH STREET, NW, SUITE 620, WASHINGTON, DC, 20005, US

(21) Appl. No.: 12/064,792

(22) PCT Filed: Sep. 12, 2006
(86) PCT No.: PCT/GB2006/003374

§ 371 Date: Aug. 1, 2008

(30) Foreign Application Priority Data
Sep. 12, 2005 (GB) 0518556.6

(51) Int. Cl. F41H 1/02; A41D 1/00; F28F 7/00; A61F 7/00; A41D 27/02

(52) U.S. Cl. 2/2.5; 2/69; 165/46; 607/96; 2/272

(57) ABSTRACT

A heat exchange system for use in a garment, for example a protective garment, or temperature conditioning garment, comprising one or more internal channels for carrying heat exchange fluid and control means for controlling the flow of the heat exchange fluid through the internal channels, wherein the internal channels are defined within a pair of sheets of weldable material by virtue of a welding process.

The invention relates to a garment to be worn by a wearer and particularly a garment capable of changing the temperature of the body of the wearer. More particularly, but not exclusively, the invention relates to a protective garment comprising body armour which is capable of heat exchange with at least a portion of the body of the wearer. Such protective garments may especially but not exclusively be useful for members of the armed forces and law enforcement organisations.

Protective garments incorporating hard armour plates, soft armour, anti-trauma padding and stab-resistant material comprising breathable fabrics are known. The purpose of breathable materials is to promote heat dissipation from the body of the wearer, thereby keeping the wearer cool, for example during physical activity. However, the effect of such garments may be dependent on the surrounding climate, and may therefore be inadequate under some conditions for ensuring the comfort of the wearer.

It is also known to provide a garment comprising cooling packs, wherein the cooling packs are generally adapted to be pre-cooled, for example by placing them in a fridge or freezer, and subsequently placed in pockets of the garment. Whilst such arrangements may provide sufficient heating or cooling, the heat exchange rate is dependent on the temperature difference between the wearer and the cooling garment. Such an arrangement may provide either excessive or insufficient cooling to the body of the wearer, thereby compromising his or her comfort. Further, the duration of use that is possible from such a system may be limited, as the temperature difference between wearer and garment decreases with use.

There also exist temperature conditioning garments in the prior art which comprise an array of pipes for carrying heat exchange fluid adapted to pass heat to, or accept heat from, a wearer. Such garments may be adapted to connect to a supply of conditioned heat exchange fluid, for example a glycol-and-water mixture from a liquid conditioning supply system installed in an aircraft. Whilst this arrangement is suitable for the applications for which it was intended, it is limited to situations where the wearer remains stationary.

Furthermore, the construction of a garment with an array of pipes is complicated.

It is an object of the heat-exchange garment of the present invention to seek to overcome, or at least to mitigate, the problems associated with the prior art.

According to a first aspect of the invention there is provided a heat exchange system for use in a garment, comprising one or more internal channels for carrying heat exchange fluid and control means for controlling the flow of the heat exchange fluid through the internal channels, characterised in that the internal channels are defined within a pair of sheets of weldable material by virtue of a welding process. Preferably, the internal channels have a substantially elliptical cross-section. More preferably, the internal channels are defined within the pair of sheets by virtue of an ultrasonic welding process.

According to a second aspect of the invention, there is provided a protective garment comprising body armour and an internal channel for carrying a flow of heat exchange fluid and control means for controlling said flow of heat exchange fluid.

Preferably, the protective garment comprises an inner layer, an outer layer and an insert, wherein the internal channel is incorporated in said insert. Preferably, body armour is incorporated in the outer layer. More preferably, the insert comprises sheets of flexible plastics material and the internal channel is formed by thermally bonding predetermined areas of those sheets of flexible plastics material. The insert may be integrally formed within the garment.

According to an optional feature of the first aspect of the invention, the protective garment comprises a container for storing heat exchange fluid. Preferably, the container is secured to the outer layer.

According to a further optional feature of the second aspect of the invention, the control means controls a pump for generating a flow of heat exchange fluid. Preferably, the pump comprises motor controlled pump adapted to variably control the flow of heat exchange fluid. More preferably, the control means comprises a microcontroller for controlling the motor controlled pump.

Preferably, the control means further comprises temperature sensing means for measuring the temperature of at least a portion of the body of a wearer. More preferably, the control means further comprises temperature sensing means for measuring the temperature of the heat exchange fluid.

According to a third aspect of the invention there is provided a temperature conditioning garment comprising an inner layer, an outer layer, at least two sheets of flexible plastics material between the inner layer and the outer layer and an internal channel for carrying a flow of heat exchange fluid, characterised in that the internal channel is formed by thermally bonding predetermined areas of the sheets of flexible plastics material. The insert may be integrally formed within the garment.

According to an optional feature of the second aspect of the invention, the garment comprises a container for storing said heat exchange fluid. Preferably, the container is secured to the outer layer.

It is preferred that the heat exchange fluid comprises water. It is further preferred that the heat exchange fluid also comprises an anti-freeze component. Optionally, that anti-freeze component may comprise a diol.

According to a further optional feature of the first aspect of the invention, the control means comprises a pump 54 for generating a flow of heat exchange fluid. Preferably, the pump comprises motor controlled pump 54 adapted to variably control the flow of heat exchange fluid. More preferably, the control means comprises a microcontroller for controlling said motor controlled pump 54.

Preferably, the control means further comprises temperature sensing means for measuring the temperature of at least a portion of the body of a wearer. More preferably, the control means further comprises temperature sensing means for measuring the temperature of the heat exchange fluid.

In order to further elucidate the description of the present invention, an exemplary non-limiting preferred embodiment will now be described with reference to the accompanying diagrammatic drawings, in which:

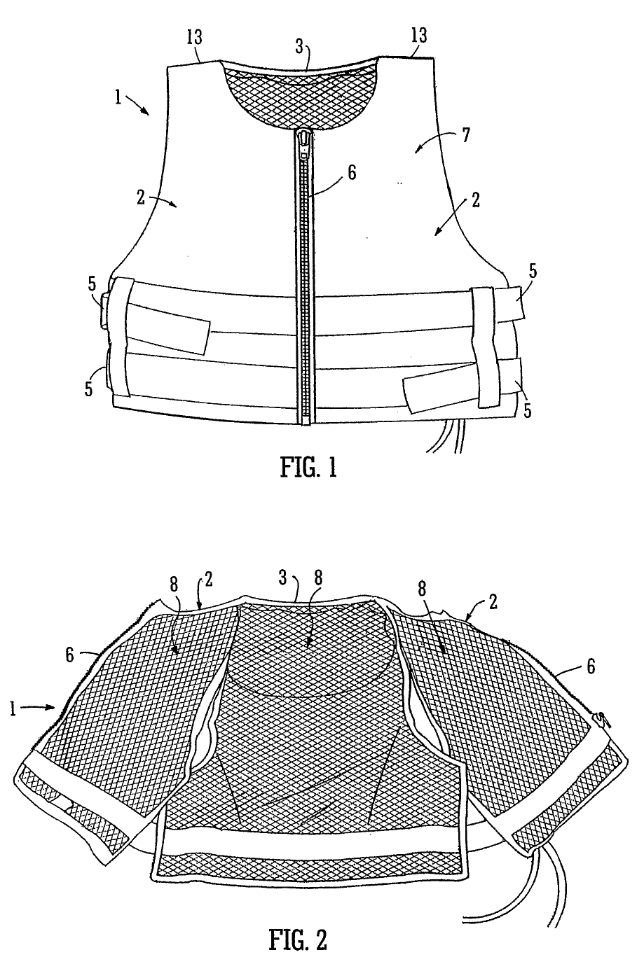

FIG. 1 is a front view of a vest according to the invention;

FIG. 2 is a front view of the vest in open condition and exposing the inner layer;

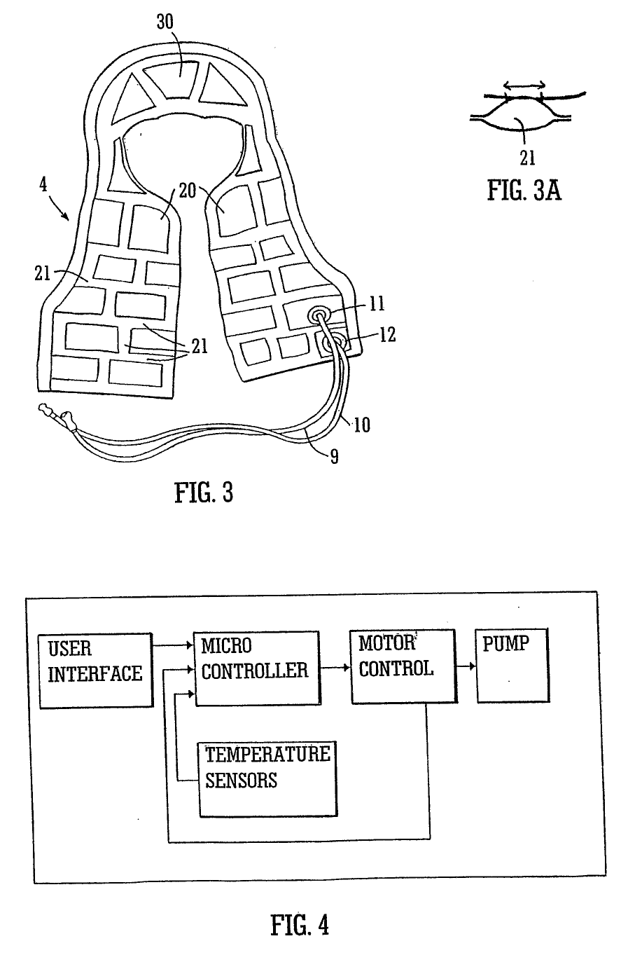

FIG. 3 is a front view of the insert;

FIG. 3A is a cross-section view of a channel within the insert;

FIG. 4 is a schematic flow-diagram illustrating the temperature control and fluid delivery system;

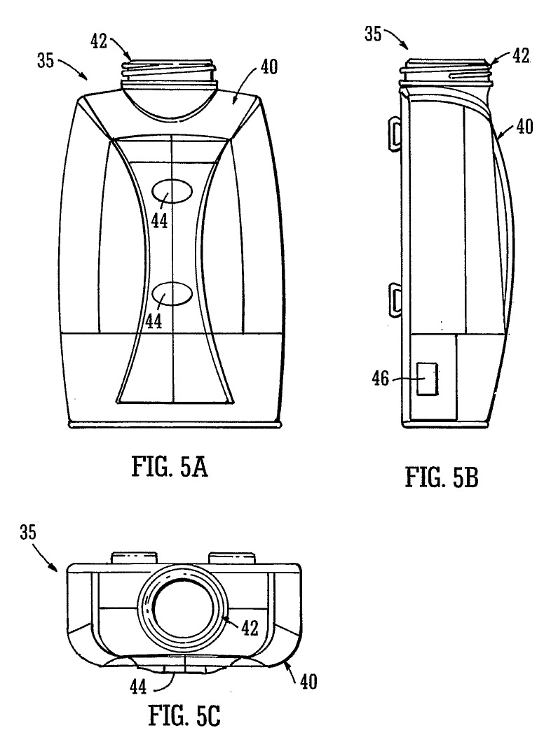

FIGS. 5a, 5b and 5c show respective front, side and plan views of the heat-exchange fluid unit 35;

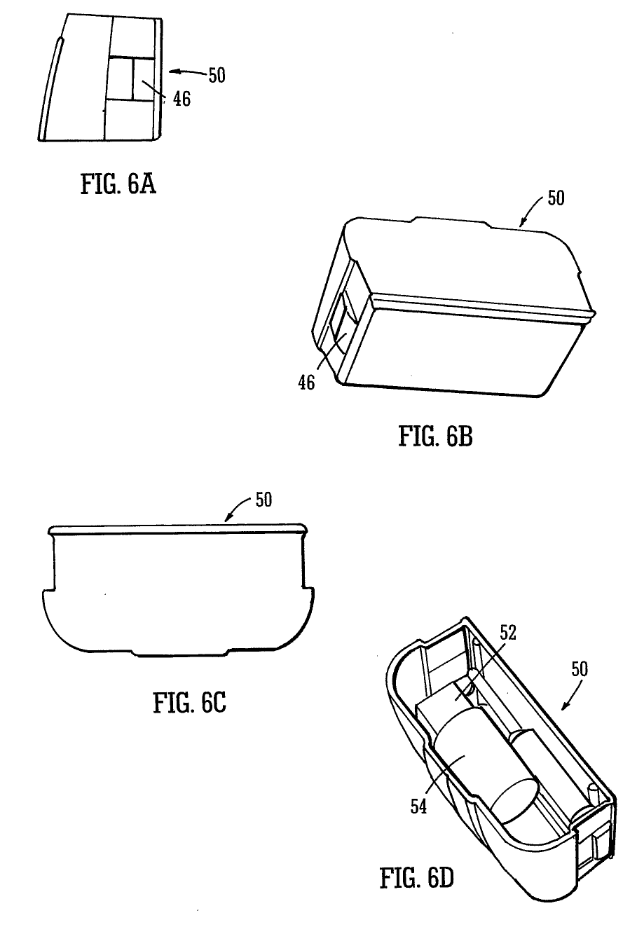

FIGS. 6a to 6d show a series of views of the control housing 50 and control means;

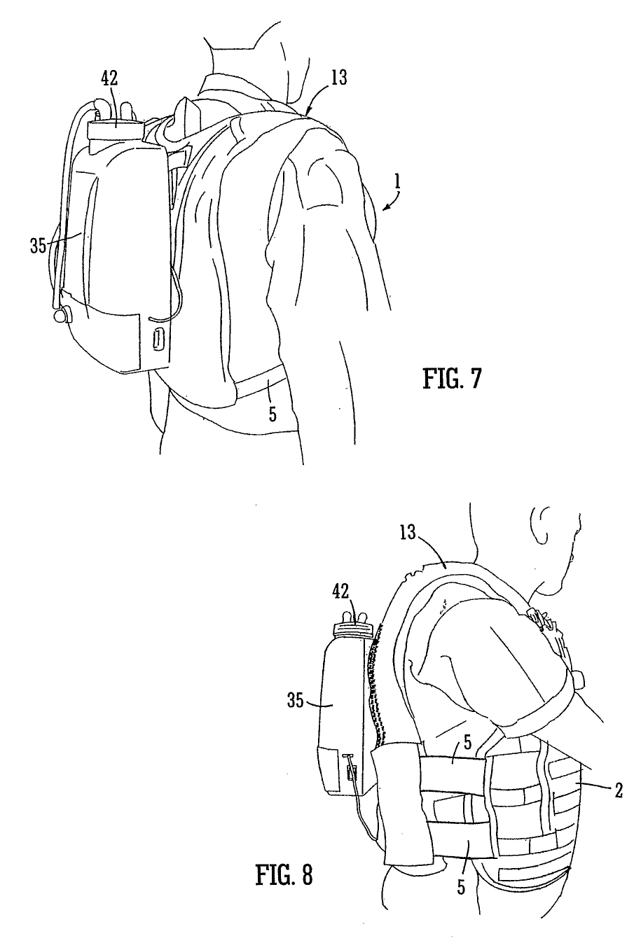

FIG. 7 is a view showing the heat exchange fluid unit worn on the back of a user;

FIG. 8 is a side view corresponding to FIG. 7;

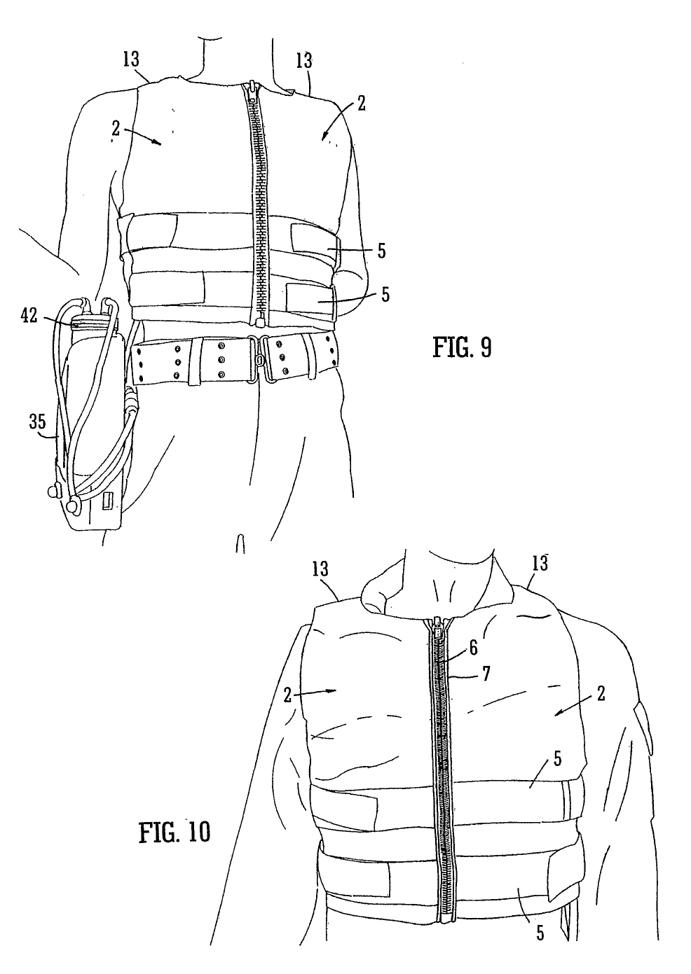

FIG. 9 is a front view showing the vest and the heat exchange fluid unit worn on the side;

FIG. 10 is a further front view of the vest;

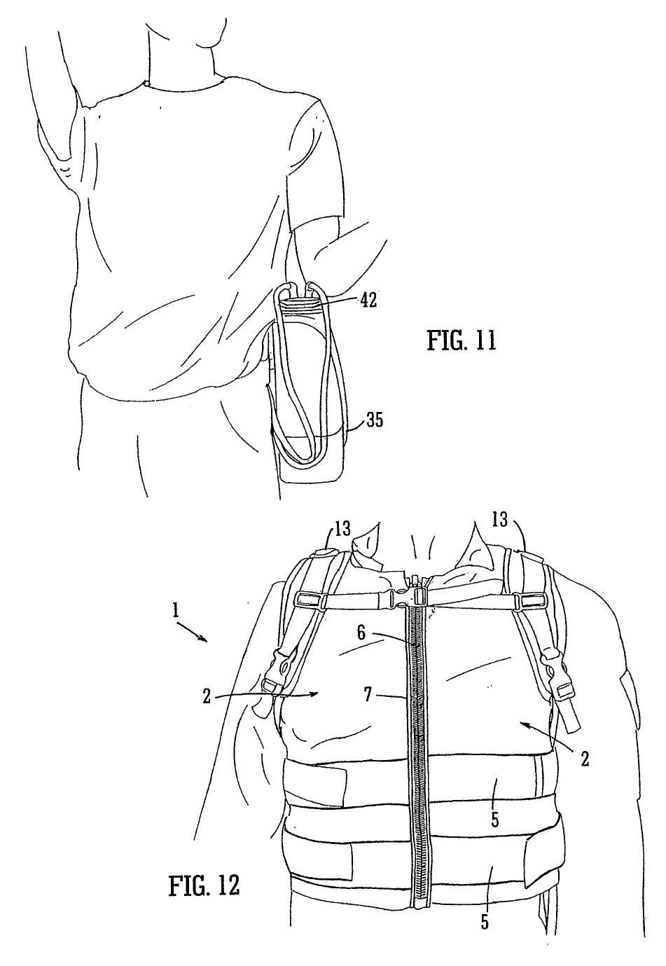

FIG. 11 just shows the side-worn heat exchange fluid unit; and

FIG. 12 is a front view of the vest with the heat exchange fluid unit worn on the back.

According to a preferred embodiment of the present invention, as shown in FIGS. 1 and 2 of the accompanying drawings, the vest comprises two front panels 2, a rear panel 3, an insert 4, Velcro straps 5 and a zip fastener 6. Each of the front panels 2 and the rear panel 3 comprises an outer layer 7 and an inner layer 8. The outer layer 7 is of known construction and, in some embodiments, incorporates body armour material (not shown) to provide resistance to bullets and stabs or to reduce trauma. The inner layer 8 comprises a known mesh material, for example polyester, to improve contact with the user.

In this embodiment, the rear panel 3 is generally rectangular in shape with its top edge comprising an arcuate portion and side edges comprise opposing arcuate portions abutting its top edge, defining part of the opening for a user's arms. The front panels 2 are mirrored in shape. Each front panel 2 is generally rectangular in shape with its top edge comprising an arcuate portion abutting a straight side edge and its side edge comprising an arcuate portion abutting its top edge, defining part of the opening for a user's arms.

The front panels 2 of the vest 1 are connected to the rear panel 3 at the top by shoulder portions 13 and at the sides by the Velcro straps 5, which provide adjustment means to accommodate a variety of waist sizes. The two front panels 2 are connected together by the zip fastener 6 wherein complementary sides of the zip fastener 6 are secured to opposing sides of the front panels 2. In assembled condition, the vest 1 comprises holes for the head, arms and body of the wearer as defined by the aforementioned arcuate shapes in the front and rear panels 2 and 3.

Intermediate the inner and outer panels 7, 8 of the front panels 2 and, preferably rear panel 3, there is provided in insert 4. The insert 4, illustrated in FIG. 3, comprises two front panel portions 20 and a rear panel portion 30. The two front panel portions are similar in shape to the front panels 2 of the vest 1 and consequently these will not be described. In this embodiment, the rear panel portion 30 is a “crescent” shape and comprises an arcuate first edge corresponding to the arcuate portion of the top edge of the rear panel 3 of the vest 1.

The insert 4 comprises two sheets of flexible plastics material which are thermally bonded together at their corresponding edges. Further regions of the sheets of flexible plastics material are thermally bonded together and define a flow channel 21. A first flexible hose 9 is connected to a first end of the flow channel 21 by a first connector 11 which is bonded to one of the sheets of plastics material in one of the front panel portions 20 of the insert 4. A second flexible hose 10 is connected to a second end of the flow channel 21 by a second connector 12 which is bonded to the same sheet of plastics material in the same front panel portion 20 of the insert 4.

The channel 21 defines a tortuous path between the first and second connectors 11 and 12. The path extends from the first front panel portion 20, through the rear panel portion 30, into the second front panel portion 20, returning through the rear panel portion 30 to the first panel portion 20 to the second connector 12.

The cross-sectional profile of the channel 21 shown in FIG. 3A is defined in such a way as to maximise the surface area disposed against the user-directed surface of the insert 4 within the vest 1. Preferably, the channel 21 is defined within the insert 4 by an ultrasonic welding process between the sheets of flexible plastics material or materials. An advantage associated with such an arrangement is that by maximising the surface area of the channel 21 defined within the insert 4 against a user-directed surface, the efficiency of heat transfer between the heat transfer fluid and the user is greatly improved.

Particularly, in prior art systems, the tubes (or pipes) have a substantially circular cross-section, having contact with the user-directed surface along only a very small fraction of its circumference. In contradistinction, the channel 21 as according to the present invention, and preferably defined by an ultrasonic welding process of the sheets of flexible plastics material which comprise the insert 4 for the vest 1, is characterised by a high-eccentricity elliptical cross-section as illustrated in FIG. 3A. In this configuration, a significantly greater fraction of its circumference is substantially in contact with the user-directed surface of the insert 4 for the vest 1. As such, heat exchange between the user and the heat-exchange fluid flowing through the channel 21 is made more efficient.

The vest 1 further comprises a container (or housing) 40, which stores a heat exchange fluid, a pump 54, a pump motor 52, a controller, a user interface and temperature sensors. The control housing 50 is of known design, is portable, light weight and releasably attachable to the vest 1. Any suitable pump 54 and pump motor means 52 may be employed within the scope of the present invention. Preferably, they will be lightweight and suitable for accurately pumping water at flow rates appropriate for this purpose.

The motor control is envisaged to use a Field Effect Transistor as a switch, using Pulse Width Modulation to enable on/off and speed control of the pump motor 52. The motor current may also be monitored to detect stall or lack of priming of the pump 54. In this preferred embodiment, the controller comprises a Peripheral Interface Controller (PIC) within the form of the microcontroller component; a typical example is presently offered under the name Microchip. Any user interface system appropriate to the needs of the user may be employed within the scope of the present invention.

According to this preferred embodiment of the present invention, the fluid delivery system comprises a heat exchange fluid unit 35, and a separate control housing 50, which control housing 50 in turn houses the motor, the controller and the battery pack for use in conjunction with the pump 54. A preferred arrangement of the heat exchange fluid unit 35 is shown in FIGS. 5a, 5b and 5c. Particularly, there is shown an outer housing 40 made of a durable water-tight material such as, for example, a plastics material.

It is preferred that, defined within that outer housing 40, there is provided one or more substantially transparent or translucent portions 44, 45 to allow the user to view the level of heat exchange fluid. Heat exchange fluid is introduced into the fluid unit 35 by virtue of access means 42 defined within the outer housing 40. There is further defined within the heat exchange fluid unit 35 according to this preferred embodiment a space for cooling means. Optionally, this cooling means is provided by an ice pack or by chilled water. In some embodiments, the outer housing 40 is adapted to be detached from the unit 35 to be placed in a freezer to cool the fluid, prior to use.

In use, the heat exchange fluid flows through or over at least part of this cooling means so as to maintain its low temperature for as long as possible. It is further preferred that the outer housing further comprises some insulation means to maintain the temperature of the cooling means and other components of the heat exchange fluid unit 35 or to minimise unwanted heating (or cooling) of the heat exchange fluid. The heat exchange fluid unit 35 is preferably adapted to be worn on the back of the user as shown in the embodiments of FIGS. 7 and 8; in an alternative embodiment, however, as shown in FIGS. 9 and 11, the heat exchange fluid unit is adapted to be worn on the user's side.

It is further preferred that the control housing 50 be removably attached to the outer housing 40 of the fluid delivery system. More preferably, this is facilitated by the provision of complementary securement means such as plastic clips 46 defined on a surface of each of the outer housing 40 and the control housing 50.

The preferred arrangement of the control housing 50 is exemplified—though not limited—by the drawings at FIGS. 6a to 6d. It contains the pump motor 52 and controller systems, and is provided with securement means which are complementary in form to those of the outer housing 40 of the heat-exchange fluid unit 35.

FIG. 4 shows a schematic diagram of the communication between the different temperature control and fluid delivery components as according to the preferred embodiment of the present invention. The parameter settings for temperature regulation are altered using the user interface, which communicates directly with the microcontroller. The microcontroller controls the pump motor 52, which in turn is coupled to the pump 54. As mentioned above, the pump motor 52 current is monitored and fed back into the microcontroller. The temperature sensors are placed in close proximity to strategic areas of the body of the wearer to provide accurate measurements of body temperature. The measured temperature is fed back into the microcontroller for closed loop temperature control.

In other embodiments, the vest or jacket may comprise sleeves and other changes to the style without departing from the scope of the present invention. The heat exchange fluid may be a water and anti-freeze component mixture, or any fluid suitable for application to a heat-exchange system.

It will be understood that directional terms such as “inner,” “outer,” “front,” “rear”, “top”, and “side” and the like serve, where used herein, merely to differentiate components of the present invention from one another; their respective components should not be considered to be limited to those orientations, and other reasonable orientations may be adopted without departing from the scope of the present invention.

1. A heat exchange system for use in a garment, comprising one or more internal channels for carrying heat exchange fluid and control means for controlling the flow of the heat exchange fluid through the internal channels, characterised in that the internal channels are defined within a pair of sheets of weldable material by virtue of a welding process.

2. A heat exchange system as claimed in claim 1, characterised in that the internal channels have a substantially elliptical cross-section.

3. A heat exchange system as claimed in claim 1 or claim 2, characterised in that the internal channels are defined within the pair of sheets by virtue of an ultrasonic welding process.

4. A protective garment comprising a heat exchange system as claimed in any one of claims 1 to 3, characterised in that the protective garment comprises an inner layer, an outer layer and an insert intermediate the inner and outer layers, the internal channels being defined within the insert.

5. A protective garment as claimed in claim 4, wherein body armour material is incorporated in the outer layer.

6. A protective garment as claimed in claim 4 or claim 5, characterised in that the insert comprises one or more sheets of flexible plastics material and the one or more internal channels are formed by thermally bonding predetermined areas of the sheets of flexible plastics material.

7. A protective garment as claimed in claim 6, characterised in that the insert is integrally formed within the garment.

8. A protective garment as claimed in any preceding claim, characterised in that the garment further comprises a container for storing the heat exchange fluid, the container being releasably secured to the protective garment.

9. A protective garment as claimed in claim 8, characterised in that the container is releasably secured to the outer layer.

10. A protective garment as claimed in any preceding claim, characterised in that the heat exchange fluid comprises water.

11. A protective garment as claimed in claim 10, characterised in that the heat exchange fluid comprises an anti-freeze component.

12. A protective garment as claimed in claim 11, characterised in that the anti-freeze component comprises a diol.

13. A protective garment as claimed in any preceding claim, characterised in that the control means controls a pump means for generating the flow of heat exchange fluid.

14. A protective garment as claimed in claim 13, characterised in that the pump means comprises motor controlled pump means adapted to variably control the flow of heat exchange fluid.

15. A protective garment as claimed in claim 13 or claim 14, characterised in that the control means comprises a microcontroller for controlling the motor controlled pump means.

16. A protective garment as claimed in any preceding claim, characterised in that the control means further comprises temperature sensing means for measuring the temperature of at least a portion of the body of a wearer.

17. A protective garment as claimed in any preceding claim, characterised in that the control means further comprises temperature sensing means for measuring the temperature of the heat exchange fluid.

18. A temperature conditioning garment comprising an inner layer, an outer layer, at least two sheets of flexible plastics material between the inner layer and the outer layer and an internal channel for carrying a flow of heat exchange fluid, characterised in that the internal channel is formed by thermally bonding predetermined areas of the sheets of flexible plastics material.

19. A temperature conditioning garment as claimed in claim 18, characterised in that the garment comprises a container for storing the heat exchange fluid.

20. A temperature conditioning garment as claimed in claim 19, characterised in that the container is secured to the outer layer.

21. A temperature conditioning garment as claimed in any one of claims 17 to 19, characterised in that control means controls a pump means for generating the flow of heat exchange fluid.

22. A temperature conditioning garment as claimed in claim 21, characterised in that the pump means comprises motor controlled pump means adapted to variably control the flow of heat exchange fluid.

23. A temperature conditioning garment as claimed in claim 22, characterised in that the control means comprises a microcontroller for controlling the motor controlled pump means.

24. A temperature conditioning garment as claimed in any one of claims 18 to 23, characterised that the control means further comprises temperature sensing means for measuring the temperature of at least a portion of the body of a wearer.

25. A temperature conditioning garment as claimed in any one of claims 18 to 24, characterised in that the control means further comprises temperature sensing means for measuring the temperature of the heat exchange fluid.
